(19) United States
(12) Patent Application Publication
(10) Pub. No.: US 2004/0209380 A1
(43) Pub. Date: Oct. 21, 2004

(54) Method for detecting a low abundance protein in a test sample

(76) Inventor: H. Bryan Bente, Landenberg, PA (US)

Correspondence Address: AGILENT TECHNOLOGIES, INC., Legal Department, DL429, Intellectual Property Administration, P.O. Box 7599, Loveland, CO, 80537-0599, US

(21) Appl. No.: 10/413,393

(22) Filed: Apr. 15, 2003

(51) Int. Cl. G01N 33/00

(52) U.S. Cl. 436/89; 422/61

(57) ABSTRACT

The present invention discloses a novel procedure of removing high abundance proteins (HAPs) from a test sample without the concurrent removal of low abundance proteins (LAPs) that are bound to the HAPs in the test sample. The procedure therefore allows the accurate detection of LAPs without the interference from the HAPs in the test sample. Specifically, the present invention provides methods for detecting LAPs in a test sample by treating the test sample with a proteolytic agent to release HAP-bound LAPs from the HAPs by fragmenting both HAPs and LAPs, removing the HAP fragments from the test sample, analyzing the LAP fragments, and identifying the LAPs in the test sample based on the characteristics of the LAP fragments. The present invention is most useful for the identification of LAPs that are bound to the HAPs in the test sample and are otherwise hard to separate from the HAPs.

### TECHNICAL FIELD

The present invention relates generally to methods of protein analysis and, in particular, to methods for detecting low abundance proteins in a test sample.

### BACKGROUND OF THE INVENTION

Protein analysis is widely used in diagnostic, medical, environmental, and industrial applications. Various analytical methods have been developed to separate and identify the proteins of interest in a test sample. Commonly used methods include gel electrophoresis, chromatography, mass spectroscopy, ELISA, immunoprecipitation, and Western blot. Generally, the proteins of interest are separated from other proteins in the test sample based on their physical properties (e.g., molecular weight, size, charge, and hydrophobicity) and/or immunological characteristics (e.g., immune-specific binding to antibodies). One common problem, however, is that the proteins of interest are often present at low levels in the test sample. The separation and identification of these low abundance proteins (LAPs) may be seriously affected by the background interference from the high abundance proteins (HAPs) in the test sample. For example, albumin and immunoglobulils are quite prevalent in serum samples and often interfere with the analysis of other serum proteins such as growth hormone or lymphokines. In order to enhance the accuracy of protein analysis of LAPs, it is desirable to diminish or completely remove the contribution of the background interference from the HAPs in the test sample.

One strategy to avoid HAP interference is to selectively remove the HAPs from the test sample using either non-specific means such as salt-precipitation, or specific means such as immuno-absorption. A major problem, however, is that many LAPs are associated with the HAPs through non-covalent interactions, such as ionic interactions, van der Waals interactions, hydrogen bonds, and hydrophobic interactions. Because these LAPs are physically bound to the HAPs, removal of the HAPs would also lead to the removal of the LAPs. The depletion of HAP-bound LAPs affects the accuracy of the protein analysis of LAPs in the test sample.

### SUMMARY OF THE INVENTION

The present invention discloses a novel procedure of removing HAPs from a test sample without the concurrent removal of HAP-bound LAPs. The procedure therefore allows the accurate detection of LAPs without the interference from the HAPs in the test sample. Specifically, the present invention provides methods for detecting LAPs in a test sample by treating the test sample with a proteolytic agent to release HAP-bound LAPs from the HAPs by fragmenting both HAPs and LAPs, removing the HAP fragments from the test sample, analyzing the LAP fragments, and identifying the LAPs in the test sample based on the characteristics of the LAP fragments. The present invention is most useful for the identification of LAPs that are bound to the HAPs in the test sample and are otherwise hard to separate from the HAPs.

In one embodiment, the present invention provides a method for detecting LAPs by treating the test sample with a proteolytic agent to fragment proteins. Typically, both the HAPs and LAPs in the test sample are degraded by the proteolytic agent. The degradation process disrupts the protein-protein interaction between LAP fragments and HAP fragments, and results in the release of the LAP fragments from the HAP fragments. The HAP fragments are removed from the sample. The unbound, LAP fragments are analyzed without the interference from the HAP fragments. The LAPs in the test sample are then identified based on the characteristics of the LAP fragments.

In another embodiment, the present invention provides a method for detecting LAPs by first isolating intact HAPs from the test sample. The HAPs, together with the LAPs that are bound to them, are then fragmented with a proteolytic agent. The fragmentation process generates both HAP and LAP fragments, disrupts protein-protein interactions between the HAP fragments and the LAP fragments, and releases the LAP fragments from the HAP fragments. The HAP fragments are removed from the reaction mixture and the unbound, LAP fragments are then analyzed without the interference of the HAPs. The LAPs that were bound to the isolated HAPs are identified based on the characteristics of the LAP fragments. In this embodiment, LAPs that do not bind to the HAPs are left in the test sample and are identified after the removal of HAPs using traditional methods.

Another aspect of the present invention pertains to a protein assay kit containing a proteolytic agent to fragment proteins for the disruption of associations between the LAPs and HAPs in a test sample, and a binding material or materials that bind specifically to the HAP fragments generated by the proteolysis of the HAPs with the proteolytic agent. Preferably, the binding material(s) is attached to a solid supporting material to facilitate the separation of the binding material(s) and the HAP fragments bound to them from other peptides in the test sample.

Other aspects of the invention will become apparent to the skilled artisan by the following description of the invention.

### BRIEF DESCRIPTION OF THE DRAWINGS

The invention of this application is better understood in conjunction with the following drawings, in which:

FIG. 1 is a flow chart describing an embodiment of the protein assay method of the present invention.

FIG. 2 is a flow chart describing another embodiment of the protein assay method of the present invention.

### DETAILED DESCRIPTION OF THE INVENTION

The following detailed description is presented to enable any person skilled in the art to make and use the invention. For purposes of explanation, specific nomenclature is set forth to provide a thorough understanding of the present invention. However, it will be apparent to one skilled in the art that these specific details are not required to practice the invention. Descriptions of specific applications are provided only as representative examples. Various modifications to the preferred embodiments will be readily apparent to one skilled in the art, and the general principles defined herein may be applied to other embodiments and applications without departing from the scope of the invention. The present invention is not intended to be limited to the embodiments shown, but is to be accorded the widest possible scope consistent with the principles and features disclosed herein.

The present invention is generally directed to methods of protein analysis and, in particular, to the identification and/or quantification of LAPs in a test sample. The present invention overcomes the problem of background interference from HAPs by digesting both LAPs and HAPs with a proteolytic agent, and removing the resulting HAP fragments prior to the analysis of the LAP fragments.

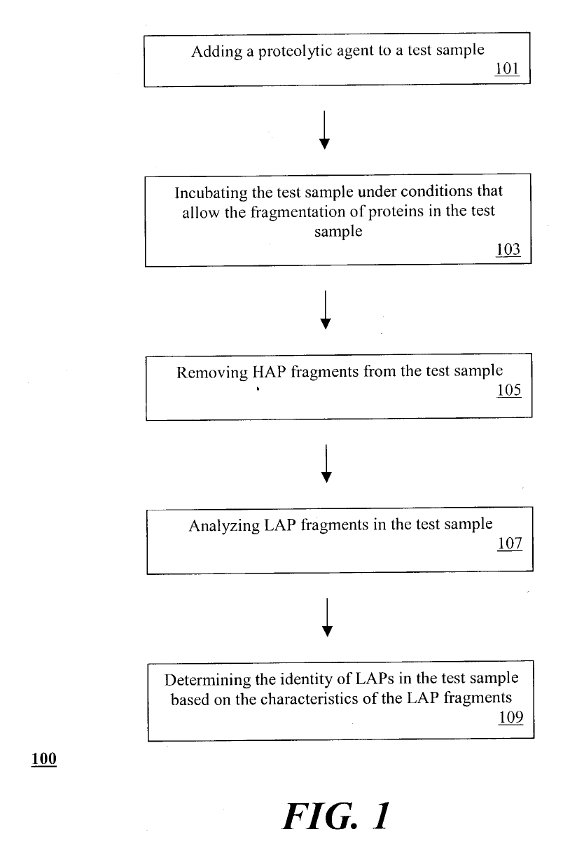

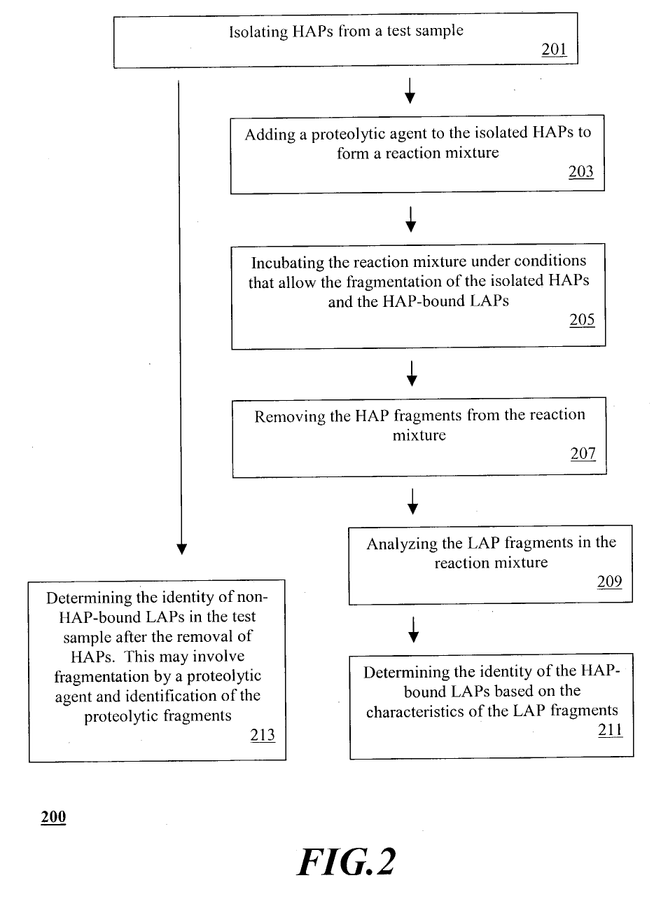

With reference now to FIGS. 1 and 2, various embodiments of the protein assay method of the present invention will be described. As will be described in more detail below, the protein assay method may be used for the identification and/or quantification of any protein of interest in a test sample.

FIG. 1 shows one embodiment of the protein assay method of the present invention, which is generally designated by the reference number 100. The first step in the method 100 is to add a proteolytic agent to a test sample (step 101). The test sample is then incubated under conditions that allow the degradation of proteins in the test sample (step 103). Typically, both HAPs and LAPs are fragmented by the proteolytic agent in step 103. The fragmentation process disrupts the protein-protein interaction between HAPs and LAPs and releases the LAPs (most likely LAP fragments after the proteolysis) from the HAPs that they associated with before the proteolysis. The HAP fragments (and any undigested HAPs) are then removed from the test sample (step 105) and the LAP fragments are analyzed without the interference from HAPs (step 107). The LAPs in the test sample are then identified (step 109) based on the characteristics of the LAP fragments analyzed in step 107.

The test sample includes, but is not limited to, biological, physiological, industrial, environmental, and other types of samples. Of particular interest are biological fluids such as serum, plasma, urine, cerebrospinal fluid, saliva, milk, broth, cell lysates, and other culture media and supernatants, as well as fractions of any of them. The test sample may also be a particular fraction of one of these samples with fractionation accomplished by one or more known methods including, but not limited to, filtration, chromatography, electrophoretic methods, or affinity methods. Physiological fluids of interest include infusion solutions, buffers, preservative or antimicrobial solutions and the like. Industrial liquids include fermentation media and other processing liquids used, for example, in the manufacture of pharmaceuticals, dairy products and malt beverages. Other sources of sample fluid which are tested by conventional methods are contemplated as within the meaning of this term as used and can, likewise, be assayed in accordance with the invention.

A protein is considered a high abundance protein (HAP), if it constitutes more than 1% by weight of total protein in a test sample. The HAPs may also be defined arbitrarily relative to a low abundance protein (LAP) or proteins. For example, in a test sample containing multiple proteins, a protein may be present in an amount that is significantly greater than the amount of another protein in the same sample. Generally, if a first protein is present in an amount that is at least three-fold of a second protein in the same sample, the first protein may be considered an HAP relative to the second protein, while the second protein may be considered an LAP relative to the first protein. In this scenario, it is possible that, in some cases, the first protein (the HAP) may amount to less than 1% of the total protein in the sample. For example, protein A amounts to 0.5% of the total protein and protein B amounts to 0.1% of the total protein in a sample. Protein A may be considered an HAP relative to protein B, althought protein A constitutes less than 1% of the total protein in the sample. It is also possible that, in some other cases, the second protein (the LAP) may amount to more than 1% of the total protein in the sample. For example, protein A amounts to 10% of the total protein and protein B amounts to 2% of the total protein in a sample. Protein B may be considered an LAP relative to protein A, althought protein B constitutes more than 1% of the total protein in the sample.

The proteolytic agent can be any agent that is capable of digesting the HAPs and releasing the HAP-bound LAPs from the HAPs. The proteolytic agent may be a protease, such as trypsin, or a mixture of proteases that hydrolyze a peptide bond between a pair of amino acids located in a polypeptide chain. The proteolytic agent may also be a chemical agent, such as cyanogen bromide (CNBr), which cleaves a peptide only on methionine residues, or a mixture of chemical agents. Many references list proteolytic agents and describe their use (for example, see Current Protocols in Protein Science, John E. Coligan, Ben M. Dunn, David W. Speicher, Paul T. Wingfield, eds. John Wiley & Sons, Inc. 1995-2001).

It should be noted that a typical test sample may contain hundreds of proteins and each protein may generate 10 to 50 fragments depending on the size of the protein and the proteolytic agent. A majority of the fragments are derived from the HAPs in the test sample. Therefore, it is necessary to remove the HAP fragments (step 105) before the characterization of the protein fragments derived from the LAPs. The removal need not to be 100%, but only to an extent that the remaining HAP fragments do not interfere with the characterization of the LAP fragments in the test sample. Accordingly, the completeness of the HAP removal in step 105 will be tailored to suit the characteristics of the particular assay system to be employed in step 107.

The removal of the HAP fragments (and any undigested HAP proteins) may be accomplished using conventional protein/peptide separation methods, which include, but are not limited to, chemical methods such as salt precipitation, physical methods such as chromatography, dialysis and filtration, and immunological methods such as affinity column and immunoprecipitation. Preferably, the HAP fragments and undigested HAPs are removed by methods based on HAP-specific immune-absorption. For example, anti-HAP antibodies may be attached to a solid supporting material, incubated with the digested test sample to allow binding of the HAP fragments to the antibodies, and removed from the test sample with the bound HAP fragments. The solid supporting material can take on a multitude of forms, such as beads, membrane, or the interior surface of a tube, vessel, or container. The solid supporting material can be mono- or multi-phasic, comprising one or more appropriate materials or mediums of similar or different absorptive or other physical characteristics. The solid supporting material can be hydrophobic or hydrophilic, bibulous or nonporous. In its most efficient embodiment, the solid supporting material is carefully tailored to suit the characteristics of the particular assay system to be employed in step 107. Methods for coating a solid supporting material with antibodies are well known in the art.

Preferably, the anti-HAP antibodies are polyclonal antibodies or a mixture of monoclonal antibodies directed to different HAPs and different fragments of the same HAP, so that the antibody-coated beads or other surfaces are capable of capturing a majority of HAP fragments and remove them from the test sample in step 105. Because different proteolytic agents result in different protein fragments, different anti-HAP fragment antibodies will be required for each proteolytic agent used.

The LAP fragments in the test sample are then analyzed in step 107. The removal of the HAP fragments and undigested HAPs in step 105 should significantly reduce the HAP interference during the sample analysis. Typically, the LAP fragments in the digested test sample are separated and characterized using conventional peptide assay methods, which include, but are not limited to, chromatography, high performance liquid chromatography, mass spectrometry, and Edman degradation. A preferred method is mass spectrometry. Again, many references exist on these techniques. Examples are High Resolution Separation and Analysis of Biological Macromolecules, Part A Fundamentals, Barry L. Karger and William S. Hancock, eds. in Methods in Enzymology, Vol. 270, Academic Press, San Diego, Calif., 1996; High Resolution Separation and Analysis of Biological Macromolecules, Part B Applications, Barry L. Karger and William S. Hancock, eds. in Methods in Enzymology, Vol. 271, Academic Press, San Diego, Calif., 1996; Mass Spectrometry of Proteins and Peptides, John R. Chapman, Ed., Humana Press, Totowa, N.J., 2000; and Current Protocols in Protein Science, John E. Coligan, Ben M. Dunn, David W. Speicher, Paul T. Wingfield, eds. John Wiley & Sons, Inc. 1995-2001.

Depending on the assay method, analysis of the LAP fragments in step 107 can be qualitative and/or quantitative. After the analysis of the LAP fragments, the identity of the LAPs in the test sample is determined based on the characteristics of the LAP fragments. For example, if the amino acid sequence of an LAP fragment has been determined in step 107, the corresponding LAP can be identified by performing a search in protein databases such as GenBank and Swiss-Prot. Similarly, protein identification may also be based on other characteristics of an LAP fragment, such as the size, electrical charge, secondary structure, and hydrophobicity of the LAP fragment. It is conceivable that a customized protein database may be constructed for the identification of LAPs using peptide characteristics determined in step 107 of the assay method 100. If the LAP fragments are quantified in step 107, it is also possible to quantify the corresponding LAPs since the amount of an LAP is proportional to the amount of the LAP fragments derived from the LAP.

FIG. 2 shows another protein assay method of the present invention, which is generally designated by the reference number 200. In the method 200, the HAPs, together with any bound LAPs, are isolated from the test sample (step 201). A proteolytic agent is added to the isolated HAPs to form a reaction mixture (step 203). The reaction mixture is incubated under conditions that allow the fragmentation of the isolated HAPs and the HAP-bound LAPs (step 205). During the fragmentation process, the proteolytic agent degrades both the HAPs and the HAP-bound LAPs, and releases the LAPs fragments from the HAP fragments. In the next step, the HAP fragments are removed from the reaction mixture (step 207). The LAP fragments in the reaction mixture are then analyzed (step 209) and the identity of the HAP-bound LAPs are determined based on the characteristics of the LAP fragments (step 211). In a parallel step, the HAP-depleted test sample is also analyzed for the LAPs that do not bind to the HAPs (step 213).

In step 201, the undigested HAPs are preferably separated from the other proteins in the test sample using an immune-absorption based method, such as affinity chromatography or immunoprecipitation. Once separation has occurred, the HAPs are released from the immune-absorption material. Similar to assay method 100, the proteolytic agent in step 203 is chosen based on the characteristics of the isolated HAPs, and the incubation conditions in step 205 are determined based on the particular proteolytic agent used in the reaction. Optimal reaction conditions for each protease or chemical proteolytic agent are well-known in the art.

The HAP fragments can be removed from the reaction mixture using conventional chemical, physical and immunological protein separation methods. Preferred separation methods include affinity chromatography, immunoprecipitation, and immune-absorption on a membrane or a solid surface. After the removal of the HAP fragments, the LAP fragments in the reaction mixture can be analyzed and the corresponding LAPs can be identified as described in assay method 100.

It should be noted that not all LAPs in the test sample are bound to the HAPs and co-isolated with the HAPs. Therefore, some LAPs are left in the test sample after the removal of the HAPs in step 201. Accordingly, the HAP-depleted test sample is also analyzed for non-HAP-bound LAPs in step 213. This analysis may involve the fragmentation of the non-HAP-bound LAPs by a proteolytic agent, the characterization of LAP fragments, and the identification of the LAPs based on the characteristics of the LAP fragments.

As understood by one skilled in the art, the methods of the present invention are most useful in detecting LAPs or LAP profiles in test samples when the identity of the LAPs are unknown before the analysis. However, it is also conceivable that the methods of the present invention can be used to quantify a known LAP or LAPs without the interference from the HAPs in the same test sample. Specifically, the methods of the present invention may be used to more accurately quantify a known LAP that binds to HAPs in a test sample. In this scenario, the LAP(s) of interest can be released from the HAPs by proteolysis, the amount of one of the free, unbound LAP fragments may be determined by an immune-absorption based method such as ELISA or RIA. The amount of the LAP of interest can then be inferred from the amount of the proteolytic fragment derived from the LAP of interest. Preferably, the HAP fragments resulting from the proteolysis are removed prior to the quantification of LAP fragments to reduce interference from the HAP fragments.

Another aspect of the present invention pertains to a detection kit for LAPs. The protein assay kit contains a proteolytic agent to fragment proteins in a test sample for the disruption of associations between the LAPs and HAPs in the test sample, and a binding material or materials that bind specifically to the HAP fragments generated by the proteolysis of the HAPs with the proteolytic agent. Preferably, the binding material(s) is attached to a solid supporting material to facilitate the separation of the binding material(s) and the peptides bound to them from other proteins/peptides in the test sample.

The preferred embodiments of novel methods for analyzing LAPs in a test sample are intended to be illustrative and not limiting. It should be understood that modifications and variations can be made by persons skilled in the art in light of the above teachings. Therefore, changes may be made in the particular embodiments disclosed which are within the scope of what is described as defined by the appended claims.

What is claimed is:

1. A method for identifying a low abundance protein in a sample, said method comprising the steps of:
(a) supplying a sample having a low abundance protein physically bound to a high abundance protein;
(b) treating the proteins with a proteolytic agent to generate proteolytic fragments from the proteins;
(c) removing proteolytic fragments of the high abundance protein from the sample; and
(d) identifying the low abundance protein using the proteolytic fragments therefrom.

2. The method of claim 1, wherein step (d) comprises:
characterizing the proteolytic fragments of the low abundance protein; and
identifying the low abundance protein using the characteristics of the proteolytic fragments therefrom.

3. The method of claim 2, wherein the proteolytic fragments of the low abundance protein are characterized using a method selected from the group consisting of chromatograpy, high performance liquid chromatography, electrophoresis, mass spectrometry, and Edman degradation.

4. The method of claim 3, wherein the proteolytic fragments of the low abundance protein are characterized using mass spectrometry.

5. The method of claim 1, wherein the proteolytic agent is a protease or a mixture of proteases.

6. The method of claim 5, wherein the proteolytic agent is trypsin.

7. The method of claim 1, wherein the proteolytic agent is a chemical agent or a mixture of chemical agents.

8. The method of claim 7, wherein the proteolytic agent is cyanogen bromide.

9. The method of claim 1, wherein the proteolytic fragments of the high abundance protein are removed using an immune-absorption method.

10. A method for identifying low abundance proteins in a sample, said method comprising the steps of:
(a) supplying a sample having low abundance proteins physically bound to high abundance proteins;
(b) isolating the high abundance proteins and the low abundant proteins bound thereto from the sample;
(c) treating the isolated proteins with a proteolytic agent to generate proteolytic fragments from the isolated proteins;
(d) removing proteolytic fragments of the high abundance proteins; and
(e) identifying the low abundance proteins using the proteolytic fragments therefrom.

11. The method of claim 10, wherein step (e) comprises:
characterizing the proteolytic fragments of the low abundance proteins; and
identifying the low abundance proteins using the characteristics of the proteolytic fragments therefrom.

12. The method of claim 11, wherein the proteolytic fragments of the low abundance proteins are characterized using a method selected from the group consisting of two-dimentional gel electrophoresis, high performance liquid chromatography, electrophoresis, column chromatograpy, mass spectrometry, and Edman degradation

13. The method of claim 12, wherein the proteolytic fragments of the low abundance proteins are characterized using mass spectrometry.

14. The method of claim 10, wherein the high abundance proteins are isolated using an immune-absorption method.

15. The method of claim 10, further comprising the step of:
identifying low abundance proteins remained in the sample after the isolation of the high abundance proteins.

16. The method of claim 15, wherein the low abundance proteins remained in the sample after the isolation of the high abundance proteins are identified by a method comprising the steps of:
treating said sample after the isolation of the high abundance proteins with a proteolytic agent to generate proteolytic fragments from the low abundance proteins;
characterizing proteolytic fragments of the low abundance proteins; and
identifying the low abundance proteins using the characteristics of the proteolytic fragments of the low abundance proteins.

17. A method for quantifying a low abundance protein in a sample, said method comprising the steps of:
supplying a sample having a low abundance protein physically bound to one or more high abundance proteins;
treating the proteins with a proteolytic agent to generate proteolytic fragments from the proteins;
quantifying proteolytic fragments of the low abundance protein; and
quantifying the low abundance protein based on the quantity of the proteolytic fragments therefrom.

18. The method of claim 17, further comprising the step of:
removing proteolytic fragments of the one or more high abundance proteins from the sample prior to the quantifaction of proteolytic fragments of the low abundance protein.

19. A kit for detecting low abundance proteins in a sample, said kit comprising:
a proteolytic agent capable of fragmenting proteins in the sample and disrupting associations between low abundance proteins and high abundance proteins in the sample; and
a binding material that binds specifically to proteolytic fragments of the high abundance proteins.

20. The kit of claim 19, wherein the binding material is attached to a solid supporting material to facilitate the removal of the binding material with bound proteolytic fragments of the high abundance proteins from the sample.
